(19) United States
(12) Patent Application Publication
(10) Pub. No.: US 2003/0220746 A1
(43) Pub. Date: Nov. 27, 2003

(54) Method and system for computing and applying a global, multi-channel background correction to a feature-based data set obtained from scanning a molecular array

(76) Inventors: Srinka Ghosh, San Francisco, CA (US); Glenda C. Delenstarr, Belmont, CA (US); Scott D. Connell, Pinckney, MI (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/153,345

(22) Filed: May 21, 2002

(51) Int. Cl. G06F 19/00; G01N 33/48; G01N 33/50

(52) U.S. Cl. 702/19

(57) ABSTRACT

A method and system for estimating a global background-signal correction for each channel of a feature-based data set, measured by a molecular array scanner, that contributes a feature-intensity data subset to the feature-based data set. The method and system of one embodiment of the present invention selects a set of features for which the measured feature intensities in two or more channels are relatively low and for which the ratio of measured feature intensities follow a central trend in a distribution of feature-intensity ratios for all features within the data set. An ideal feature is computed from the selected set of low-intensity features, from which separate global, residual background-signal corrections for each channel can be calculated and applied to that channel's feature-intensity data subset within the feature-based data set.

### TECHNICAL FIELD

The present invention relates to the analysis of data obtained by scanning molecular arrays and, in particular, to a method and system for determining a multi-channel, global, background-signal-intensity correction for a data set comprising feature-signal magnitudes obtained from scanning a molecular array previously exposed to labeled target molecules.

### BACKGROUND OF THE INVENTION

Nothing in the following discussion is admitted to be prior art unless specifically identified as “prior art.” The present invention is related to processing of data scanned from arrays. Array technologies have gained prominence in biological research and are likely to become important and widely used diagnostic tools in the healthcare industry. Currently, molecular-array techniques are most often used to determine the concentrations of particular nucleic-acid polymers in complex sample solutions. Molecular-array-based analytical techniques are not, however, restricted to analysis of nucleic acid solutions, but may be employed to analyze complex solutions of any type of molecule that can be optically or radiometrically scanned and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. The subunit molecules for RNA include: (1) adenosine, abbreviated “A,” a purine nucleoside; (2) uracil, abbreviated “U,” a pyrimidine nucleoside; (3) cytosine, abbreviated “C,” a pyrimidine nucleoside; and (4) guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer. In RNA polymers, the nucleotides contain ribose sugars rather than deoxy-ribose sugars. In ribose, a hydroxyl group takes the place of the 2′ hydrogen 128 in a DNA nucleotide. RNA polymers contain uridine nucleosides rather than the deoxy-thymidine nucleosides contained in DNA. The pyrimidine base uracil lacks a methyl group (130 in FIG. 1) contained in the pyrimidine base thymine of deoxy-thymidine.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by confornational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrates the hydrogen bonding between the purine and pyrimidine bases of two anti-parallel DNA strands. FIG. 2A shows hydrogen bonding between adenine and thymine bases of corresponding adenosine and thymidine subunits, and FIG. 2B shows hydrogen bonding between guanine and cytosine bases of corresponding guanosine and cytosine subunits. Note that there are two hydrogen bonds 202 and 203 in the adenine/thymine base pair, and three hydrogen bonds 204-206 in the guanosine/cytosine base pair, as a result of which GC base pairs contribute greater thermodynamic stability to DNA duplexes than AT base pairs. AT and GC base pairs, illustrated in FIGS. 2A-B, are known as Watson-Crick (“WC”) base pairs.

Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304. The ribbon-like strands in FIG. 3 represent the deoxyribose and phosphate backbones of the two anti-parallel strands, with hydrogen-bonding purine and pyrimidine base pairs, such as base pair 306, interconnecting the two strands. Deoxy-guanylate subunits of one strand are generally paired with deoxy-cytidilate subunits from the other strand, and deoxy-thymidilate subunits in one strand are generally paired with deoxy-adenylate subunits from the other strand. However, non-WC base pairings may occur within double-stranded DNA.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex. Strictly A-T and G-C complementarity between anti-parallel polymers leads to the greatest thermodynamic stability, but partial complementarity including non-WC base pairing may also occur to produce relatively stable associations between partially-complementary polymers. In general, the longer the regions of consecutive WC base pairing between two nucleic acid polymers, the greater the stability of hybridization between the two polymers under renaturing conditions.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 4-6, the principle of array-based hybridization assays is illustrated with respect to the single feature 404 to which a number of identical probes 405-409 are bound. In practice, each feature of the array contains a high density of such probes but, for the sake of clarity, only a subset of these are shown in FIGS. 4-6.

Once an array has been prepared, the array may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contains nucleotide sequences complementary to any of the probes bound to array surface do not hybridize to generate stable duplexes and, as a result, tend to remain in solution. The sample solution is then rinsed from the surface of the array, washing away any unbound-labeled DNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for scanning. Biotin and avidin represent an example of a pair of chemical moieties that can be utilized for such steps.

Finally, as shown in FIG. 6, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the array as shown in FIG. 7, with features to which labeled target molecules are hybridized similar to 706 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

Array-based hybridization techniques allow extremely complex solutions of DNA molecules to be analyzed in a single experiment. An array may contain from hundreds to tens of thousands of different oligonucleotide probes, allowing for the detection of a subset of complementary sequences from a complex pool of different target DNA or RNA polymers. In order to perform different sets of hybridization analyses, arrays containing different sets of bound oligonucleotides are manufactured by any of a number of complex manufacturing techniques. These techniques generally involve synthesizing the oligonucleotides within corresponding features of the array through a series of complex iterative synthetic steps.

One, two, or more than two data subsets within a data set can be obtained from a single molecular array by scanning the molecular array for one, two or more than two types of signals. Two or more data subsets can also be obtained by combining data from two different arrays. When optical scanning is used to detect fluorescent or chemiluminescent emission from chromophore labels, a first set of signals, or data subset, may be generated by scanning the molecular at a first optical wavelength, a second set of signals, or data subset, may be generated by scanning the molecular at a second optical wavelength, and additional sets of signals may be generated by scanning the molecular at additional optical wavelengths. Different signals may be obtained from a molecular array by radiometric scanning to detect radioactive emissions one, two, or more than two different energy levels. Target molecules may be labeled with either a first chromophore that emits light at a first wavelength, or a second chromophore that emits light at a second wavelength. Following hybridization, the molecular array can be scanned at the first wavelength to detect target molecules, labeled with the first chromophore, hybridized to features of the molecular array, and can then be scanned at the second wavelength to detect target molecules, labeled with the second chromophore, hybridized to the features of the molecular array. In one common molecular array system, the first chromophore emits light at a red visible-light wavelength, and the second chromophore emits light at a green, visible-light wavelength. The data set obtained from scanning the molecular array at the red wavelength is referred to as the “red signal,” and the data set obtained from scanning the molecular array at the green wavelength is referred to as the “green signal.” While it is common to use one or two different chromophores, it is possible to use three, four, or more than four different chromophores and to scan a molecular array at three, four, or more than four wavelengths to produce three, four, or more than four data sets.

FIG. 8 shows a small region of a scanned image of a molecular array containing an image of a single feature. In FIG. 8, the small region of the scanned image comprises a grid, or matrix, of pixels, such as pixel 802. In FIG. 8, the magnitude of the signal scanned from the small region of the surface of a molecular array spatially corresponding to a particular pixel in the scanned image is indicated by a kind of gray scaling. Pixels corresponding to high-intensity signals, such as pixel 804, are darkly colored, while pixels having very low signal intensities, such as pixel 802, are not colored. The range of intermediate signal intensities is represented, in FIG. 8, by a generally decreasing density of crosshatch lines within a pixel. In FIG. 8, there is a generally disc-shaped region in the center of the region of the scanned image of the molecular array that contains a high proportion of high-intensity pixels. Outside of this central, disc-shaped region corresponding to a feature, the intensities of the pixels fall off relatively quickly, although pixels with intermediate intensities are found, infrequently, even toward the edges of the region of the scanned image, relatively distant from the obvious central, disc-shaped region of high-intensity pixels that corresponds to the feature.

In general, data sets collected from molecular arrays comprise an indexed set of numerical signal intensities associated with pixels. The pixel intensities range over the possible values for the size of the memory-storage unit employed to store the pixel intensities. In many current systems, a 16-bit word is employed to store the intensity value associated with each pixel, and a data set can be considered to be a 2-dimensional array of pixel-intensity values corresponding to the 2-dimensional array of pixels that together compose a scanned image of a molecular array.

FIG. 9 shows a 2-dimensional array of pixel-intensity values corresponding to a portion of the central, disc-shaped region corresponding to a feature in the region of a scanned image of a molecular array shown on FIG. 8. In FIG. 9, for example, pixel intensity 902 corresponds to pixel 806 in FIG. 8.

Features on the surface of a molecular array may have various different shapes and sizes, depending on the manufacturing process by which the molecular array is produced. In one important class of molecular arrays, features are tiny, disc-shaped regions on the surface of the molecular array produced by ink-jet-based application of probe molecules, or probe-molecular-precursors, to the surface of the molecular array substrate. FIG. 10 shows an idealized representation of a feature, such as the feature shown in FIG. 8, on a small section of the surface of a molecular array. FIG. 11 shows a graph of pixel-intensity values versus position along a line bisecting a feature in the scanned image of the feature. For example, the graph shown in FIG. 11 may be obtained by plotting the intensity values associated with pixels along lines 1002 or 1004 in FIG. 10. Consider a traversal of the pixels along line 1002 starting from point 1006 and ending at point 1008. In FIG. 11, points 1106 and 1108 along the horizontal axis correspond to positions 1006 and 1008 along line 1002 in FIG. 10. Initially, at positions well removed from the central, disc-shaped region of the feature in 1010, the scanned signal intensity is relatively low. As the central, disc-shaped region of the feature is approached, along line 1002, the pixels intensities remain at a fairly constant, background level up to point 1012, corresponding to point 1112 in FIG. 11. Between points 1012 and 1014, corresponding to points 1112 and 1114 in FIG. 11, the average intensity of pixels rapidly increases to a relatively high intensity level 1115 at a point 1014 coincident with the outer edge of the central, disc-shaped region of the feature. The intensity remains relatively high over the central, disc-shaped region of the feature 1116, and begins to fall off starting at point 1018, corresponding to point 1118 in FIG. 11, at the far side of the central, disc-shaped region of the feature. The intensity rapidly falls off with increasing distance from the central, disc-shaped region of the feature until again reaching a relatively constant, background level at point 1008, corresponding to point 1108 in FIG. 11. The exact shape of the pixel-intensity-versus-position graph, and the size and shape of the feature, are dependent on the particular type of molecular array and molecular-array substrate, chromophore or a radioisotope used to label target molecules, experimental conditions to which the molecular array is subjected, the molecular-array scanner used to scan a molecular array, and on data processing components of the molecular-array scanner and an associated computer that produce the scanned image and pixel-intensity data sets. For example, with some type of array manufacture processes or with different hybridization and washing protocols, the features may resemble donuts, or even more irregular blobs.

The background signal generated during scanning regions of the surface of a molecular array outside of the areas corresponding to features arises from many different sources, including contamination of the molecular-array surface by fluorescent or radioactively labeled or naturally radioactive compounds, fluorescence or radiation emission from the molecular-array substrate, dark signal generated by the photo detectors in the molecular-array scanner, and many other sources. When this background signal is measured on the portion of the array that is outside of the areas corresponding to a feature, it is often referred to as the “local” background signal.

An important part of molecular-array data processing is a determination of the background signal that needs to be subtracted from a feature. With appropriate background-subtraction, it is possible to distinguish low-signal features from no-signal features and to calculate accurate and reproducible log ratios between multi-channel and/or inter-array data. The sources of background signal that appear in the local background region may be identical to the sources of background signal that occur on the feature itself; that is, the signal represented in the local background region may be additive to the signal that arises from the specific labeled target hybridized to probes on that feature. In this case, it is appropriate to use the signal from the local background region as the best estimate of the background to subtract from that feature.

FIG. 12 illustrates a currently employed technique for measuring the local background signal for a feature. FIG. 12 corresponds to the small region of the scanned image of a molecular array shown on FIG. 8. Initial pixel-based coordinates for the center of the feature can be estimated from manufacturing data for the molecular array and from a number of scanned-image processing techniques. Using these initial pixel-based coordinates for the center of the feature, the integrated intensities of disc-shaped regions with increasing radii centered at those coordinates can be computed to determine, by a decrease in integrated intensities, the outer edge 1202 of the central, disc-shaped feature region. An intermediate region, in which the integrated pixel intensities rapidly fall off with increasing radius, corresponding to the regions in FIG. 11 between points 1112 and 1114 and between points 1118 and 1108, can be determined to provide the outer boundary 1204 of a region of interest (“ROI”) surrounding and including the central, disc-shaped region of the feature. Finally, an annulus lying between the outer edge of the ROI 1204 and a somewhat arbitrary outer background circumference 1206 is considered to be the background region for the feature, and the integrated intensity of this background region 1208, divided by the area of the background region, is taken to be the background signal for the entire region comprising the feature region, feature ROI, and background annulus. Alternatively, the locations and sizes of feature regions may be known in advance of the image processing stage, based on array manufacturing data and other information, and so the ROI may not need to be determined by a method such as the method described above. Thus, a current technique for background signal estimation is based on a local method involving determining an integrated signal intensity for an annulus surrounding the ROI disc associated with a feature, and determining a background-signal intensity per image area. The estimated local background signal for a feature is the background-signal intensity per image area, and is subtracted from the normalized raw feature signal, to produce a background-subtracted feature signal. A feature-based data set includes background-subtracted data subset, for each signal scanned, comprising feature signals or raw feature signals.

In general, for a large class of molecular-array-based experiments, the logarithm of the ratio of two feature signals, measured in two channels, for each feature, referred to as the log ratio, is calculated as a measure of the differential concentrations of target molecules in those two sample solutions that bind to the feature. A plurality of channels may include both intra-array channels, two or more signal channels scanned from a single array, and may also include inter-array channels, one or more signal channels scanned from two or more arrays. Unfortunately, when both signals are relatively weak, or of low magnitude, the log ratio can be extremely sensitive to slight perturbations in the relative weak intensities of the two signals. Imprecise background signal correction can lead to anomalous log ratios and spurious results based on the anomalous log ratios. Manufacturers and designers of molecular arrays and molecular array scanners, as well as researchers and diagnosticians who use molecular arrays in experimental and commercial settings, have recognized the need for accurate background subtraction methods, in order to obtain more accurate and reproducible gene expression data.

### SUMMARY OF THE INVENTION

One embodiment of the present invention provides a method and system for estimating a global, background-signal correction for each channel, measured by a molecular array scanner, that contributes a feature-intensity data subset to a feature-based data set. The method and system of one embodiment of the present invention selects a set of features for which the measured feature intensities in two or more channels are relatively low and for which the ratio of measured feature intensities follow a central trend in a distribution of feature-intensity ratios for all features within the data set. A characteristic background feature is computed from the selected set of low-intensity features, from which separate global, residual background-signal corrections for each channel can be calculated and applied to that channel's feature-intensity data subset within the feature-based data set.

### BRIEF DESCRIPTION OF THE DRAWINGS

FIG. 1 illustrates a short DNA polymer.

FIG. 2A shows hydrogen bonding between adenine and thymine bases of corresponding adenosine and thymidine subunits.

FIG. 2B shows hydrogen bonding between guanine and cytosine bases of corresponding guanosine and cytosine subunits.

FIG. 3 illustrates a short section of a DNA double helix.

FIGS. 4-7 illustrate the principle of array-based hybridization assays.

FIG. 8 shows a small region of a scanned image of a molecular array containing the image of a single feature.

FIG. 9 shows a 2-dimensional array of pixel-intensity values corresponding to a portion of the central, disc-shaped region corresponding to a feature in the region of a scanned image of a molecular array shown on FIG. 8.

FIG. 10 shows an idealized representation of a feature, such as the feature shown in FIG. 8, on a small section of the surface of a molecular array.

FIG. 11 shows a graph of pixel-intensity values versus position along a line bisecting a feature in the scanned image of the feature.

FIG. 12 illustrates a currently employed technique for measuring the background signal for a feature.

FIG. 13 illustrates a currently available approach to residual, global background signal correction of a two-channel, feature-based data set.

FIGS. 14-15 illustrate low-intensity anomalies that occur in graphically analyzed data sets resulting from incorrect or imprecise global background-signal correction by the technique shown in FIG. 13.

FIG. 16 illustrates a better approach to correcting residual background signals.

FIG. 17 illustrates an over-corrected data set resulting from local background-signal subtraction.

FIG. 18 is a high-level flow diagram describing the method of one embodiment of the present invention.

FIGS. 19A-B show a feature having uniform intensity distribution and a feature have non-uniform intensity distribution.

FIG. 20 illustrates the central trend within a data set.

FIGS. 21A-B, 22A-B, and 23-24 illustrate a rank-order method for selecting features from a set of filtered features.

FIG. 25 illustrates the fitting of a line to the low-combined-intensity central-trend features.

FIG. 26 illustrates the principle of the MAD technique.

FIGS. 27-28 illustrate two methods that can be employed to filter the low-combined-intensity central-trend features with respect to the fitted line.

FIGS. 29-30 illustrate the construction of an ideal background data point.

FIG. 31 illustrates a final refinement technique for calculating the ideal characteristic background feature.

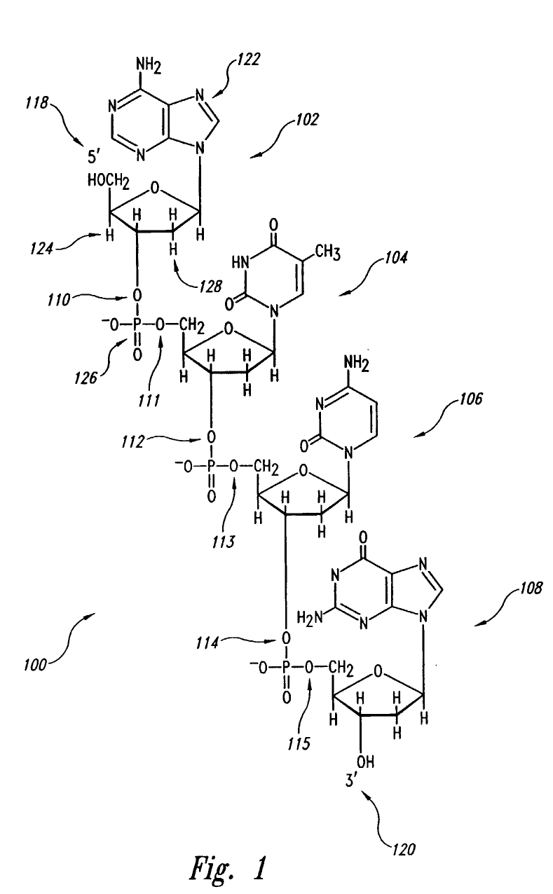

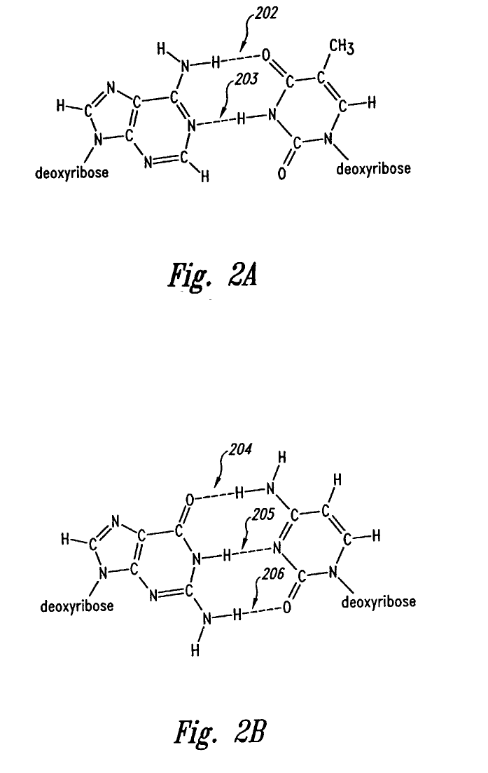

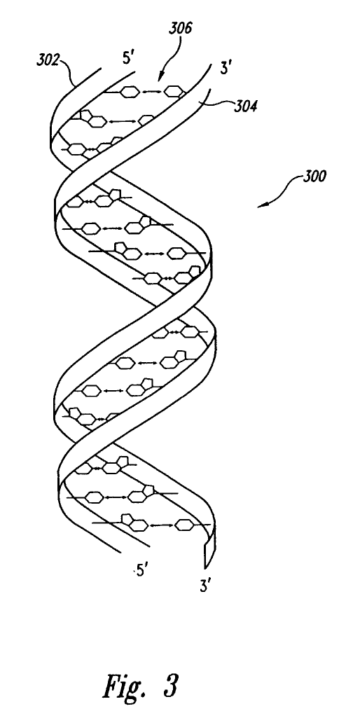

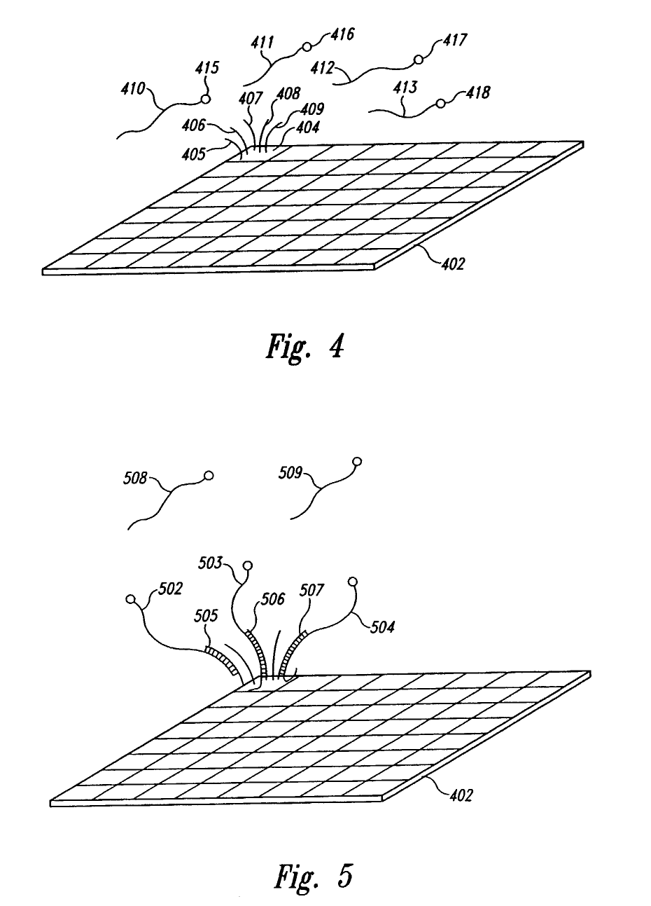

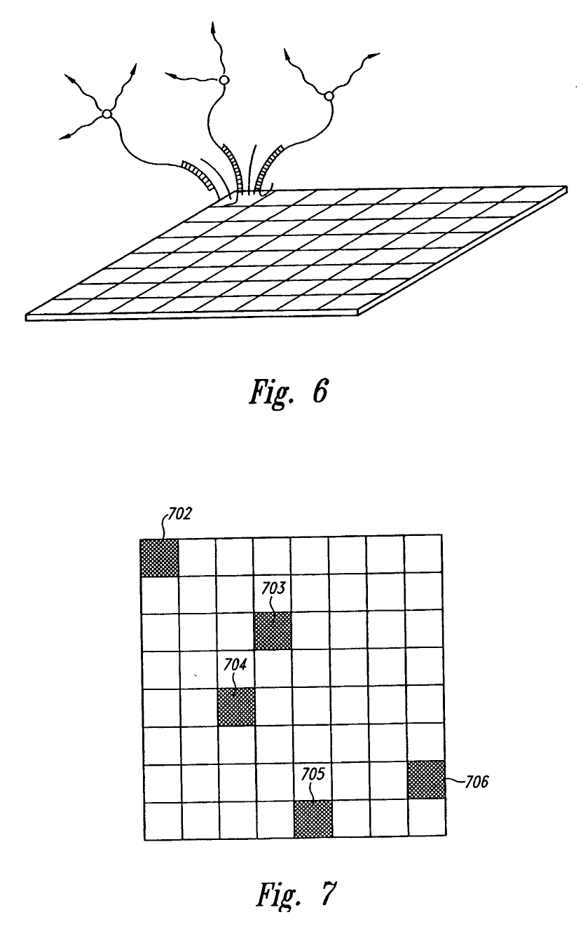

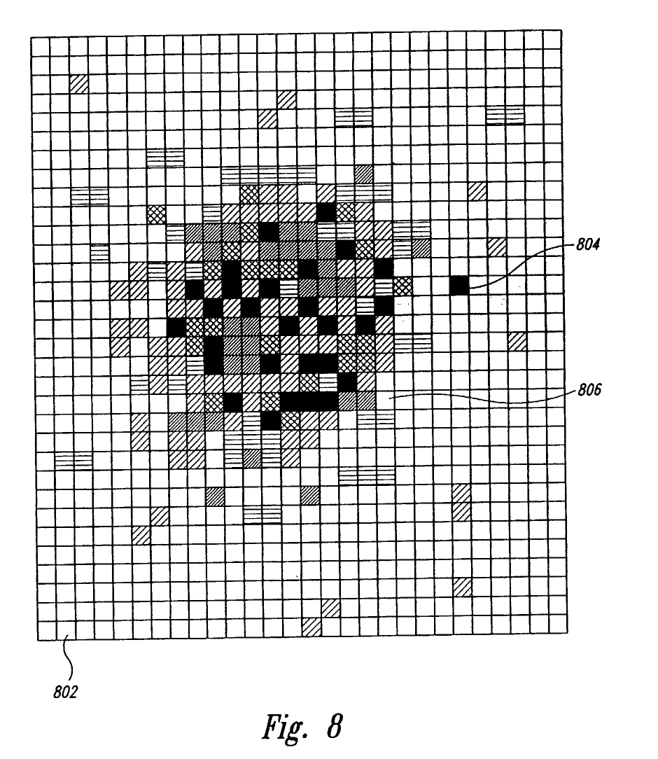

### DETAILED DESCRIPTION OF THE INVENTION

One embodiment on the present invention is directed to method and system for determining global-background-signal-based corrections for each channel in a feature-based, molecular-array data set. In one embodiment, described below, the method provides separate global, residual-background-signal corrections for each of two channels C1 and C2. This method is useful for current molecular-array-based experiments in which two different chromophores, radioactive labels, or other types of labels are employed and scanned in two different channels in a molecular array scanner. However, the present invention is not restricted to determining global, residual-background-signal corrections for two channels, but can be extended to additional channels when more than two labels are employed in molecular-array-based experiments and detected by a molecular array scanner. The present invention is not restricted to correcting data from a single array, or intra-array data. There are two broad categories of potential corrections for data obtained from multiple arrays, or inter-array data. The first category involves two arrays that are each single-channel arrays, and a need to correctly compare C1 signal from a first array with C1 signal from a second array. The second category involves multi-channel arrays, and a need, for example, to compare C1 signal from a first array with C2 signal from a second array. The present invention is not restricted to correcting data from background-subtracted signals. An embodiment of the method of the present invention can be applied to raw signals, before any background-subtraction is done, and thus includes both background-signal subtraction and a global, residual background correction method in a single step.

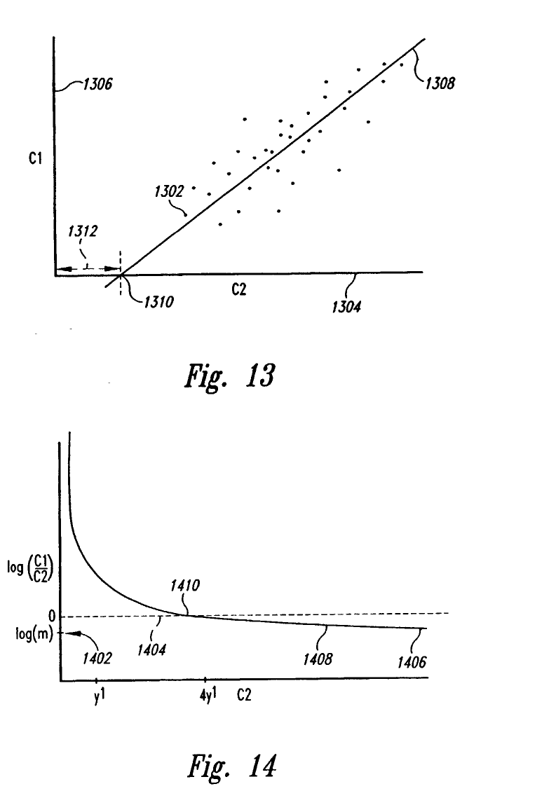

The described embodiment of the present invention is directed to identifying C1 and C2 residual background-signal corrections. When the C1-signal and C2-signal intensities of each feature are plotted in a C1/C2 plot, the data points corresponding to features are generally distributed about a central-trend line or curve. In general, there is an apparent cluster of smallest-intensity features at a low-intensity region of the distribution, or lowest-intensity point of the central-trend line or curve. In the described embodiment, the C1 and C2 residual background-signal corrections are equivalent to the x′ and y′ coordinates for an ideal, characteristic, low intensity data point within the cluster of smallest-intensity features in a C1/C2 plot. The C1 and C2 feature intensities for the features in the data set can be corrected to translate the ideal, characteristic, low intensity data point to the origin of the C1/C2 plot, or equivalently, the magnitudes of the C1 and C2 residual background-signal corrections correspond to the x′ and y′ coordinates for the ideal, characteristic, low intensity data point in a C1/C2 plot. One embodiment of the present invention is described in four subsections that follow: (1) additional information about molecular arrays; (2) an overview of the method of one embodiment of the present invention, presented with reference to FIGS. 13-17; (3) a flow-control-diagram and illustration-based description of the method of the embodiment of the present invention; and (4) a Matlab-like pseudocode implementation of the embodiment.

### Additional Information about Molecular Arrays

An array may include any one-, two- or three-dimensional arrangement of addressable regions, or features, each bearing a particular chemical moiety or moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Interfeature areas are typically, but not necessarily, present. Interfeature areas generally do not carry probe molecules. Such interfeature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used such as described in U.S. Pat. Nos. 5,599,695, 5,753,788, and 6,329,143. Interfeature areas need not be present particularly when the arrays are made by photolithographic methods as described in those patents.

A molecular array is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent applications Ser. No. 10/087447 “Reading Dry Chemical Arrays Through The Substrate” by Corson et al., and Ser. No. 09/846125 “Reading Multi-Featured Arrays” by Dorsel et al. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. Nos. 6,251,685, 6,221,583 and elsewhere.

A result obtained from reading an array, followed by application of a method of the present invention, may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based molecular array, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a molecular array by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Molecular array experiments can also be used to diagnose disease, for gene sequencing, and for analytical chemistry. Processing of molecular-array data can produce detailed chemical and biological analyses, disease diagnoses, and other information that can be stored in a computer-readable medium, transferred to an intercommunicating entity via electronic signals, printed in a human-readable format, or otherwise made available for further use.

### Overview

In some cases, it is more appropriate to use a global method to determine the background signal and then to subtract that global background signal from all features of an array. One example is to calculate an average or median of all the local background regions on an array. Another method is to determine a minimum feature or local-background-region signal. Another method is to use a set of features, referred to as “negative controls,” that are designed to not bind labeled target in a specific manner.

To determine the correctness of a background subtraction method, it is common to analyze data from self-self arrays. A self-self experiment generally involves taking one sample and splitting it for labeling. A first portion of the sample is labeled with a first label and an equal, second portion of the sample is labeled with a second label. The two portions are then applied to a single array for hybridization, washing, and scanning. Three types of graphical analysis of such an array should yield the following three results, respectively, after correct background subtraction: (1) a C1/C2 plot plotted linearly in both dimensions is generally approximately linear and symmetric; (2) a C1/C2 plot plotted in log scale is generally approximately linear and symmetric; and (3) a plot of the log of the ratio C1/C2 versus C1 or C2 should be approximately horizontal. The slope of the lines from first and second graphical analyses, described above, and the y-intercept from the third graphical analysis, described above, reflect a constant that is needed for dye-normalization. Such a constant reflects different efficiencies in labeling for the C1 and C2 labels and different efficiencies in scanner optics. Once the data is dye-normalized, the data generally yields a slope of 1.0 for the C1/C2 plots and a y intercept of 0 for the plot of the log of the ratio C1/C2 versus C1 or C2. For the purpose of this discussion, the dye-normalization constant can be applied to features in a signal-independent fashion. In many observed cases, dye-normalization is often signal-dependent and curve-fitting may be needed to achieve optimal results. Indeed, for cases where a curve-fitting algorithm is employed for dye-normalization, the results will be more robust if correct background subtraction has first been applied. When background-signal correction is incorrect or imprecise, the ideal, expected results from the above-described graphical analyses, and expected results for similar analytic approaches for the curve-fitting techniques needed for non-linear cases, are not observed. Instead, the graphical analyses produce anomalous trends and features.

FIG. 13 illustrates one approach to residual, global background signal correction of a two-channel, feature-based data set. FIG. 13 is a C1/C2 plot of the features in a hypothetical data set. In FIG. 13, the horizontal x axis 1304 corresponds to the intensity of a feature in the C2 channel, and the vertical y axis 1306 corresponds to the feature intensity in the C1 channel. In FIG. 13, a data point, such as data point 1302, is plotted with xy coordinates equal to a corresponding feature's C2-signal and C1-signal intensities. The data points corresponding to features often form a cloud-like distribution about a line or curve fit, by well-known mathematical curve-fitting techniques, to the distribution. For example, in FIG. 13, a line 1308 has been fit to the distribution of points. C2 and C1 may represent feature intensities measured at green and red wavelengths, respectively; or may represent feature intensities measured at the same wavelength from two different arrays, or feature intensities measured for two different labels via radiometric, magnetic, or electrical scanning techniques

In general, the feature intensities within a data set span a relatively large range of values, from very high intensities down to a lowest set of intensities generally somewhat above the 0-level intensity, for non-background-subtracted signals. In the case of background-subtracted signals, the lowest set of signals may have negative values. In a self-self experiment, the data points should fall symmetrically about a line with a slope that corresponds to a constant C1/C2 ratio that reflects the relative target labeling efficiencies and relative efficiencies by which the C1 and C2 signals are measured by a molecular array scanner. Again, as noted above, the number of C1 and C2 labeled target molecules resident within any particular feature in a molecular array produced in a self-self experiment should be nearly identical. Even in a differential expression experiment, the distribution of data points generally exhibits a central trend, to which a line or curve can be mathematically fit.

In one currently employed background-signal correction technique, the best-fit line, 1308 in FIG. 13, is extended until it intersects with the x axis or y axis. For example, in FIG. 13, the best-fit line is extended downward and leftward to the point 1310, the x-axis intercept. The data subset corresponding to the axis intercepted, in the case of FIG. 13, the x axis corresponding to the C2 data subset, is then corrected by applying the magnitude of the x-axis correction 1312 to the data subset to translate the intersection point 1310 to the origin of the C1/C2 plot. In general, the sign of the x or y coordinate of the intercept is reversed, and the x or y coordinate added to the feature signals. However, this technique is inadequate. In general, residual background signals are present in both C1 and C2 data subsets, so that the relative position of the distribution of data points is offset in both x and y directions with respect to the coordinate system. Moreover, extending the central-trend curve to an x-axis or y-axis intersection is an arbitrary construct, without sound theoretical basis.

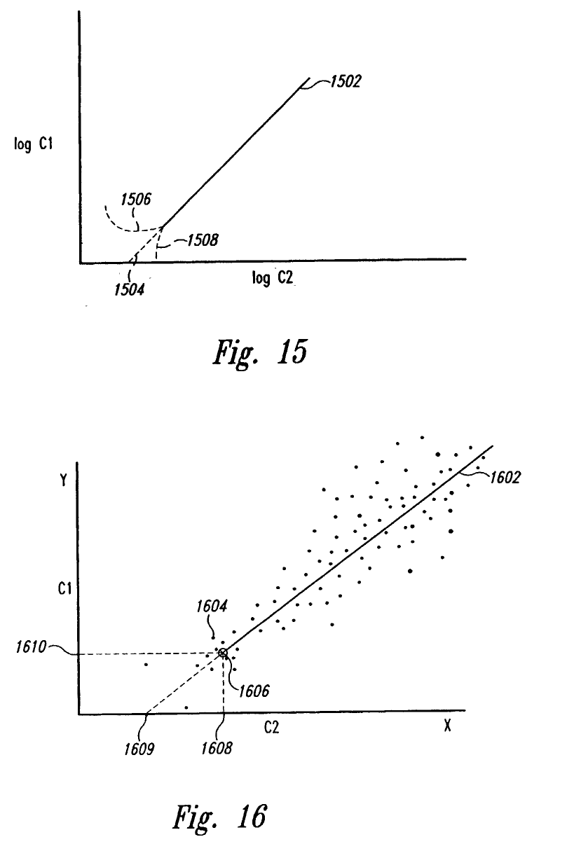

FIGS. 14-15 illustrate low-intensity anomalies that occur in graphically analyzed data sets resulting from incorrect or imprecise global background-signal correction by the technique shown in FIG. 13. FIG. 14 shows a representation of a log-ratio-versus-C2-signal intensity plot for features of a data set. Note that, in FIG. 14, and in many subsequent figures, a curve is shown to represent the central trend of a distribution of data points that would be plotted from an actual data set. The data set plotted in FIG. 14 would produce a C1/C2 plot similar to that shown in FIG. 13, with a C1/C2 best-fit line having a slope less than 1. A log-ratio-versus-C2-signal intensity plot would be expected to produce an essentially horizontal, or constant, line, offset by the value log(m) (1402 in FIG. 14), where m is the slope of the C1/C2 best-fit line in a C1/C2 plot, from the horizontal axis 1404. For large signal intensity ratios, the log-ratio-versus-C2-signal intensity plot does approximate a horizontal line with the expected offset from the x axis, as at data point 1406. However, as the combined-signal feature intensity decreases, toward the y axis, the log-ratio-versus-C2-signal intensity central-trend curve begins to dramatically curve upward, crossing the x axis at point 1410, and asymptotically approaching the y axis for very small values. Plots of log(C1) versus log (C2) exhibit similar, low-intensity anomalies. As shown in FIG. 15, a log(C1)-versus-log-(C2) is also expected to show a linear central-trend line for a data set with a relatively constant C1/C2 ratio. But, for low intensity features, the central-trend line 1502, rather than continuing 1504 at a constant slope towards an x-axis or y-axis intercept, is often observed to curve away from line 1502 in either a positive direction 1506 or negative direction 1508.

An important contribution to the anomalous, low-intensity departure of observed central-trend curves from expected central-trend curves for log-ratio-versus-C2-signal and log(C1)-versus-log-(C2) plots, discussed above with reference to FIGS. 14 and 15, is the flawed approach to global, residual background correction, noted above with reference to FIG. 13. If, after global, residual background correction, residual background signals remain within the data set, and if the residual background signals are of different magnitudes for the two channels, then, for low intensity features, as the feature intensity in one channel approaches the corrected 0-signal level, the feature intensity in the other channel may approach the value of the difference between the residual background signals in the two channels. The log ratio then begins to rapidly depart from a general, constant value, veering non-linearly either towards plus infinity or towards minus infinity. In general, the upwards curving seen in FIG. 14 and FIG. 15 occurs when C1 is under-corrected for background signal, relative to C2, and downwards curving, as seen in FIG. 15, occurs when C2 is under-corrected for background signal, relative to C1.

FIG. 16 illustrates. a better approach to correcting residual background signals. In FIG. 16, a central-trend line 1602 is fit to a distribution of points, such as point 1604, in a C1/C2 plot. However, in the better approach, instead of arbitrarily extending the central-trend line towards an x-axis or y-axis intercept, the location of an ideal, characteristic, low intensity data point 1606 is estimated based on the lowest intensity, non-outlying data points clustered about the central-trend line. The x and y coordinates 1608 and 1610 of this ideal, characteristic, low intensity data point represents C2 and C1 global residual background-signal corrections. Application of the C2 and C1 global residual background-signal corrections has the effect of moving the position of the ideal, characteristic, low intensity data point to the origin of the C1/C2 plot. Unlike the technique described with respect to FIG. 13, in which one data subset is shifted relative to the other data subset, the better approach illustrated in FIG. 16 involves calculating both C2 and C1 global residual background-signal corrections, based upon perpendicular projections to the axes rather than a linear extrapolation, and applying both C2 and C1 global residual background-signal corrections to their respective data subsets. The location of the ideal, characteristic, low intensity data point is a good approximation for a data point corresponding to an ideal, no-background-signal-in-either-channel feature, and its relative position with respect to the x and y axes reflects the respective global, residual background signals in the C2 and C1 data subsets. By contrast, the x-axis intersection 1608 of the central-trend line is reflective of neither the C2 nor C1 global residual background-signals.

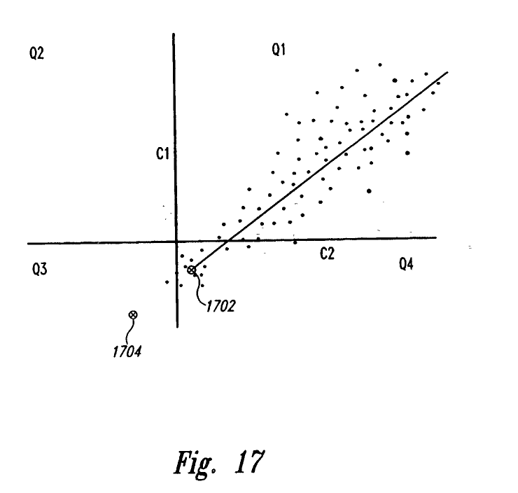

It should be noted that global, residual background signals in the C2 and C1 data subsets may be positive or negative for local-background-subtracted data sets. Local-background subtraction may lead to over-correction in one or both channels, in which case positive global residual background-signal corrections may need to be applied in one or both channels. FIG. 17 illustrates an over-corrected data set resulting from local background-signal subtraction. In FIG. 17, the ideal, characteristic, low intensity data point 1702 falls within the fourth quadrant, below the x axis. In this case, a positive C1 global, residual background-signal correction and a negative C2 global residual background-signal correction need to be applied to the data set in order to bring the ideal, characteristic, low intensity data point 1702 to the origin of the C1/C2 plot. Had the location of the ideal, characteristic, low intensity data point fallen in the third quadrant 1704, then positive C1 and C2 global residual background-signal corrections would need to be applied to the data set.

Note that, in quasi-self-self experiments, in which multiple 1-channel arrays are exposed to the same sample solution and scanned, C1/C1′ plots can be employed to identify the position of an ideal, characteristic, low intensity data point to allow for calculation of separate, global, residual background-signal corrections for the C1 and C1′ data subsets. In both self-self, and quasi-self-self experiments, the distribution of data points about a central-trend line tends to be symmetrical. In differential experiments, although not fully symmetrical, the distribution nonetheless clusters about a central-trend line or curve and the subject of this invention will provide an effective background correction method. A quasi-differential expression experiment can result from comparing a signal set from one array with one sample type versus a second array hybridized with a second sample type. This can occur with either single or multi-channel array data. The subject of this invention will also provide an effective background correction method for these cases.

### Flow-Control Diagram and Graphical Description of One Embodiment

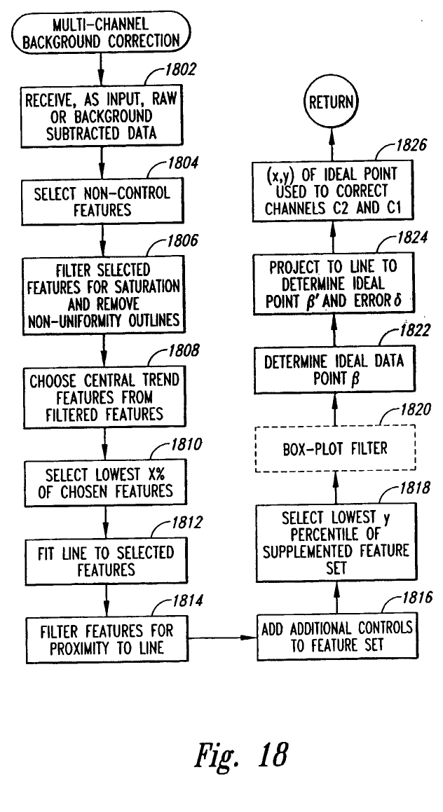

FIG. 18 is a high-level flow diagram describing the method of one embodiment of the present invention. This method will be described with reference to FIG. 18 and concurrent reference to subsequent figures that provide greater detail for many of the steps shown in FIG. 18.

In the first step 1802 of the two-channel background correction method, a two-channel, feature-based molecular-array data set is received. As discussed earlier, this data set can comprise two data subsets corresponding to different channels of one array, or this data set can comprise two data subsets corresponding to signals obtained from different arrays. The feature intensities of the data set can be either raw intensities or local-background-subtracted intensities. Next, in the step 1804, those features in the data set not marked as control features are selected. Initially, control features are filtered out, or rejected, because they may not to exhibit C1/C2 ratios close to the central trend for C1/C2 ratios within the data set.

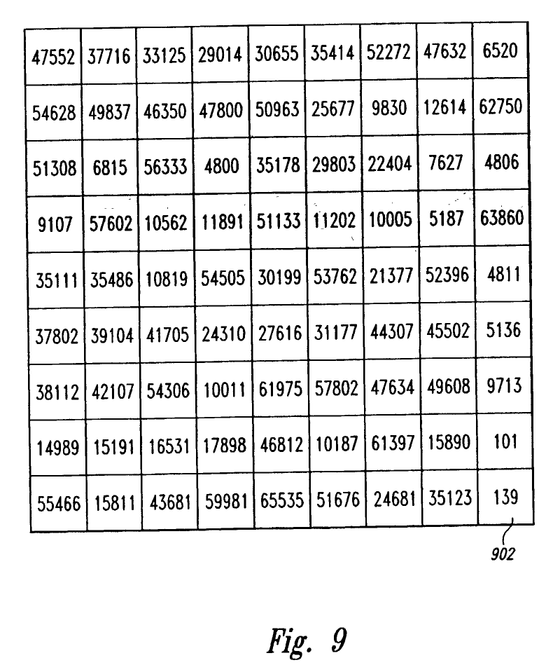

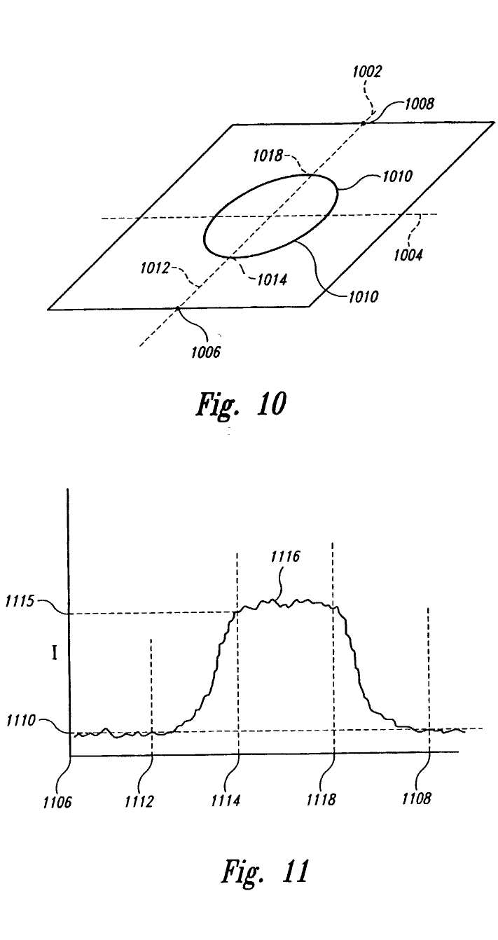

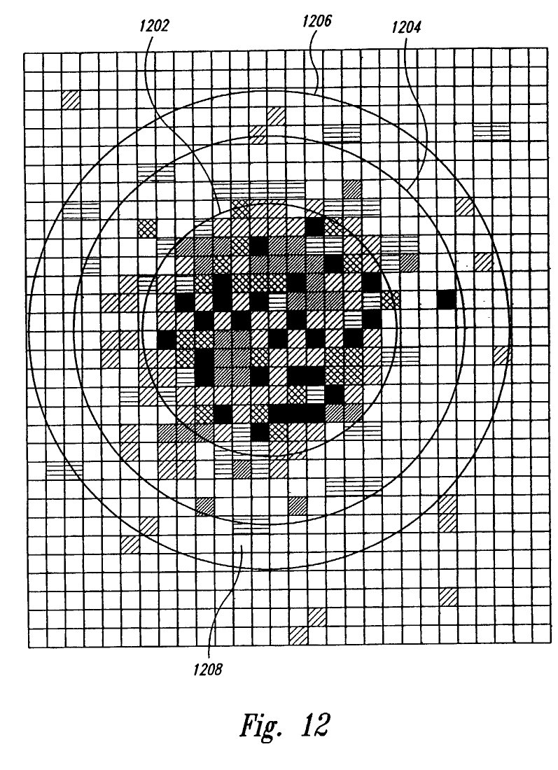

In step 1806, the selected non-control features are filtered to remove features with a saturation level above a threshold saturation level and features with non-uniform pixel-intensity distributions. As discussed above with reference to FIG. 9, the scanned image of a molecular array consists of a 2-dimensional array of pixel-intensity values, commonly stored in 16-bit words, and therefore ranging from 0 to 65535. A pixel having an intensity value of 65535 is considered to be saturated, because all measured intensity values greater that 65335 are encoded as the maximum value 65535. When more than a threshold percentage of the pixels within the area corresponding to a feature are saturated, then the feature is considered to be saturated. In other words, the true intensity of the feature is not reflected in the intensity value integrated over the pixels within the feature area. In one embodiment of the present invention, the saturation-level threshold is 5% of the feature pixels, and features having more than 5% saturated pixels are removed from the set.

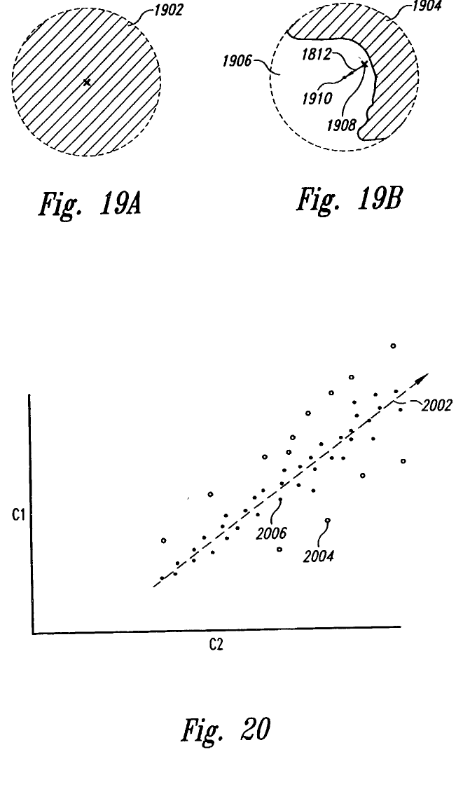

A second filter, carried out in step 1806, is designed to remove features with non-uniform intensity distributions over the area of the feature. FIGS. 19A-B show a feature having uniform intensity distribution and a feature have non-feature 1902 is shown with cross-hatching indicating a uniform distribution of moderate intensity values over the entire area of the image of the feature. By contrast, in FIG. 19B, a crescent-shaped portion 1904 of the area of a feature has medium intensity values, while the remaining portion of a feature, 1906, has low intensity values. The signal intensities within the feature shown in FIG. 19B are non-uniformly distributed. Non-uniform distribution of intensities can be detected in a number of different of ways. The statistical variance of pixel intensities within the area of image of a feature can be computed, and features with pixel-intensity variances greater than a threshold variance can be considered to have non-uniform pixel-intensity distributions. The threshold may be a function of biological or electronic noise, or noise on the microarray due to chemical or hybridization processes, for example. Alternatively, as shown in FIG. 19B, a centroid for the pixel intensity distribution 1908 can be computed, and the location of the centroid compared to the location of the geometric center 1910 of the image of the feature. When the distance 1912 between the centroid of pixel-intensity and the geometric center is greater than a threshold distance value, the feature can be considered to have a non-uniform pixel-intensity distribution. Alternatively, both the mean and the median signal statistics can be computed and a feature can be considered to have a non-uniform pixel-intensity distribution if the difference between the mean signal and the median signal exceeds a threshold or if the difference divided by either the mean or the median signal exceeds a threshold. Many other alternative techniques can be employed in order to classify features having uniform and non-uniform pixel-intensity distributions. In step 1806, features having non-uniform pixel distributions are removed from the selected set of non-control features. As noted above, features in certain types of arrays may not be disc-shaped, and techniques used to compute a metric of uniformity may need to be tailored specifically to the particular feature shapes present in the arrays.

In step 1808, features that fall within a central trend within the data set are chosen from the set of filtered features produced in step 1806. Such features exhibit generally an equal, negligibly different, relation between the signals in the two channels.

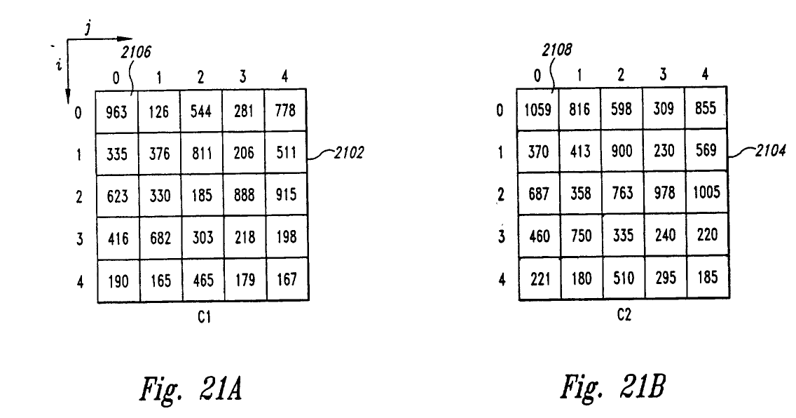

FIG. 20 illustrates the central trend within a data set. In FIG. 20, as in FIGS. 13, 16, and 17, described above, each feature is plotted with respect to the features' signal-intensities in the C2 and C1 channels. Features close to the line 2002 are features having a C1/C2 ratio close to a characteristic C1/C2 ratio for the data set. Features further from the line, such as feature 2004, have C1/C2 ratios that markedly depart from the characteristic C1/C2 ratio for the data set. In differential experiments, a certain portion of the data points are expected to fall outside of close proximity to the central-trend, reflecting different signals arising from different concentrations of target molecules in the two sample solutions, and the overall distribution of data points may be somewhat asymmetrical. However, even in arrays with differential expression, a set of features following a central tendency can be found. In self-self and quasi-self-self experiments, a symmetrical distribution relatively closely following a linear central trend is expected. In step 1808, the method of the present invention seeks to select those features, such as feature 2006, with C1/C2 ratios that follow the characteristic C1/C2 ratio for the data set. The distribution of C1/C2 ratios need not correspond to a linear distribution shown in FIG. 20, but may exhibit a more complex central trend, that might, for example, be mathematically described as a second-order or greater-order polynomial function of the C1/C2 ratios with respect to C2-signal intensity.

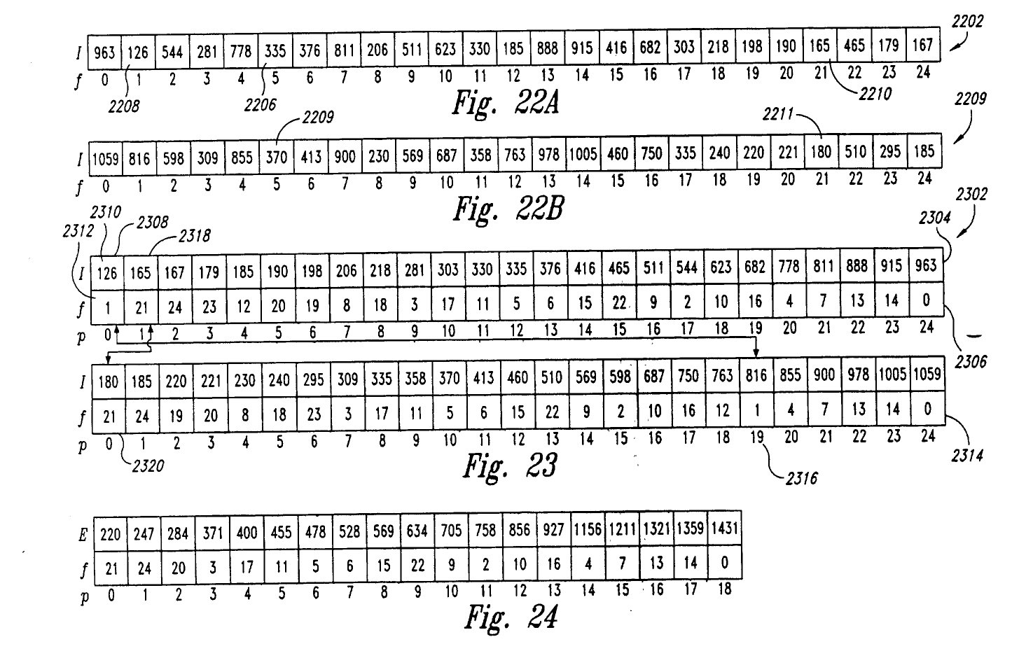

FIGS. 21A-B and 22-24 illustrate a rank-order method for selecting features, from the set of filtered features produced in step 1806, that follow the central trend in C1/C2 ratios of the features of the data set. In FIGS. 21A-B, the C1 and C2-signal intensities for a small set of 25 features is shown. Each feature is described by indices i and j that designate the position of the feature intensity for the feature within each of two 2-dimensional arrays 2102 and 2104 containing feature intensities for a particular channel. Thus, for example, feature (0,0) has the C1-signal intensity 963 (2106 in FIG. 21A) and the C2-signal intensity 1059 (2108 in FIG. 21B).

The C1 and C2-signal intensities for the 25 features can be alternatively considered to reside in two 1-dimensional arrays. FIGS. 22A-B shows the C1 and C2 signal intensities, stored in the 2-dimensional arrays of FIGS. 21A-B, alternatively considered to be stored in two 1-dimensional arrays, each with a single index f The feature signals are indexed in row order, with respect to the 2-dimensional arrays, within the 1-dimensional arrays. Thus, the feature signals for feature (1,0) are stored in the sixth elements 2206 and 2209 of the 1-dimensional arrays 2202 and 2204 in FIGS. 22A-B.

The two sets of feature signals within the data set are next ranked with respect to signal intensity. In FIG. 23, two 2-dimensional arrays corresponding to the two 1-dimensional arrays in FIG. 22 contain the C1 and C2 signal intensity data contained in the 1-dimensional arrays in FIG. 22. In the 2-dimensional array 2302, corresponding to 1-dimensional array 2202 in FIG. 22, the first row 2304 contains sorted feature intensities, and the second row 2306 contains the corresponding indices of the feature in the 1-dimensional array 2202 in FIG. 22. For example, the feature with the lowest C1 intensity is contained in the second element 2208 of the 1-dimensional array 2202 in FIG. 22 with index “1,” and is stored the first column 2308 of 2-dimensional array 2302 in FIG. 23, with the entry 2310 in the first row of the first column containing the feature intensity “126” and the entry 2312 in the second row in the first column containing the index “1” of the feature 2208 from the 1-dimensional array 2202 in FIG. 22. The index p of the column in a 2-dimensional array (2302 or 2314) containing a feature intensity and its associated 1-dimensional array index corresponds to the rank of the feature within the feature-signal data subset stored in the 2-dimensional array and sorted in ascending order with respect to intensity. Thus, feature “1” with intensity “126,” being the lowest intensity feature in the C1-signal data subset, is stored in the first element of array 2302 with ρ=0 and therefore has rank “0.” Array 2302 contains the sorted C1-signal data subset, and the array 2314 contains the sorted C2-signal subset.

In order to find those features having a C1/C2 ratio close to the characteristic C1/C2 ratio for the data set, or, in other words, those features close to the central trend within the data set, the ranks of a feature in the two sorted subsets contained in arrays 2302 and 2314 are compared. Those features having identical or similar ranks in both data subsets are considered to lie within the central trend of the C1/C2 distribution for the data set. For example, feature “1” (2208 in FIG. 22A) has rank “0” in the C1 data subset (2308 in FIG. 23A) and has rank “19” (2316 in FIG. 23B) in the C2 data subset. Feature “1” is therefore not considered to be a central-trend feature. By contrast, feature “21” (2210-2211 in FIGS. 22A-B, 2318 in FIG. 23A, and 2320 in FIG. 23B) has ranks “1” and “0” in the sorted C1 and C2 data subsets, respectively. Feature “21” is therefore considered to be a central-trend feature within the C1/C2 distribution. More specifically, features are considered to be central-trend features when the relative rank displacement is less than a threshold value s, as expressed in the following expression:

$$\frac{\vert {ρ}_{C1} - {ρ}_{C2} \vert}{( max {ρ}_{C1} - min {ρ}_{C1} )} \leq s$$

where ρ_{C1} is the rank of C1 signal intensity in the sorted C1 data subset, and ρ_{C2} is the rank of C2 signal intensity in the sorted C2 data subset. The denominator in the above case can also be considered as the total number of features. In this context, the above formula can be generalized as the normalized relative rank displacement, where the sum of features is the normalization parameter.

The threshold s can be either arbitrarily chosen, or, in an alternative embodiment, is chosen based on the correlation coefficient for the C1/C2 distribution of the features of the data set. The correlation coefficient is provided by the following equation:

$$\frac{{[ {\sum }^{N} ( C1 - \overline{C1} ) ( C2 - \overline{C2} ) ]}^{2}}{{{[ {\sum }^{N} C1 - \overline{C1} )}^{2} ] [ {\sum }^{N} C2 - \overline{C2} )}^{2} ]}$$

where \overline{C1} is the average C1-signal intensity, \overline{C2} is the average C2-signal intensity, N is the number of features in the data set. Alternatively, the correlation coefficient can be based on absolute distances from the central trend curve, on vertical distances from the central trend curve, or on any number of other distribution-based density or dispersion metrics.

The selected central-trend features are then sorted according to a combined feature intensity E given by the following expression:

$$E = \sqrt{{( C1 )}^{2} + {( C2 )}^{2}}$$

In fact, the features can be sorted by metric of similarity. In case of applying the described mechanism to features with background subtracted intensity, it is necessary to maintain a distinction between features with resultant negative versus positive signals. Therefore, prior to the evaluation of the E metric described above, it is necessary to translate the origin(0,0), such that all features populate the positive quadrant only. In other words, the coordinate axes are moved in order to reposition the ideal, characteristic, low intensity data point, shown in third and fourth quadrant positions in FIG. 17, into the first, positive quadrant. This mechanism is implemented solely to compute E and does not introduce any perturbation into the true data set. FIG. 24 shows the central-trend features selected via the ranking process described with reference to FIGS. 21A-B, 22A-B, and 23 ranked in ascending order with respect to the combined feature intensity E computed with respect to the above formula. Note that, each feature in the central-trend, combined-feature-intensity data subset stored in the array shown in FIG. 24 is associated with a rank.

In step 1810, the method selects, as chosen features, a lowest combined-feature-intensity portion of the central-trend, combined-feature-intensity features. A threshold-percentile cutoff, x, for choosing the lowest-combined-intensity features may have a specific, fixed value, or maybe tunable with respect to additional constraints, such as the absolute number of features within the chosen percentile range and other constraints. At the end of step 1810, a set of low-combined-intensity, central-trend features have been selected. In one embodiment, the threshold x is determined by starting with an x value that includes nearly all central-trend features, and iteratively reduces x until the slope of the best-fit line and a distribution metric stabilize, assuming that the distribution of data points around the central-trend yields a curve, rather than a line, or alternatively, increases x from a low starting point until the slope of the best-fit line and a distribution metric become non-stable.

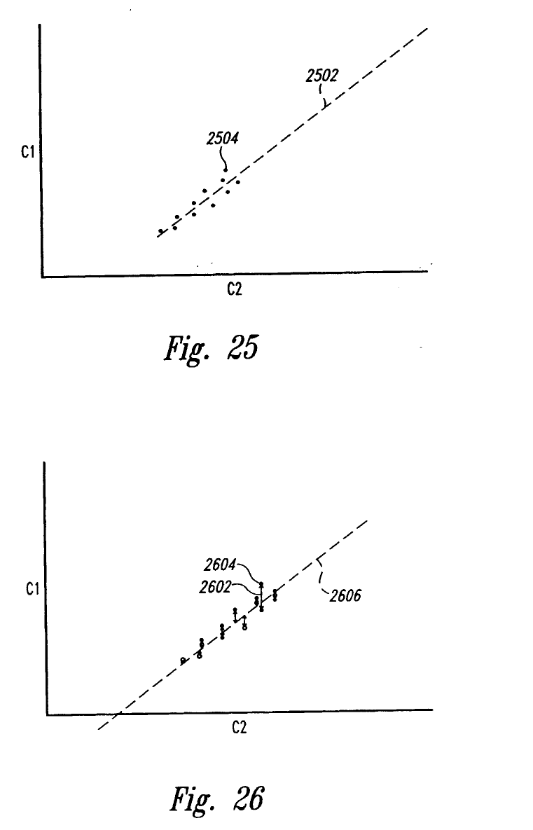

In step 1812, a line is fit to the C1/C2 distribution of the low-combined-intensity central-trend features. FIG. 25 illustrates the fitting of a line to the low-combined-intensity, central-trend features. In FIG. 25, the best-fit line 2502 is seen to represent the trend in the distribution of the plotted features, such as 2504. Many different possible line-fitting techniques can be employed. In a preferred embodiment, a technique that minimizes the mean or median absolute deviation (“MAD”) is employed. In one minimization technique, the following quantity M is minimized:

$$M = {\sum }_{i = 1}^{N} \vert {y}_{i} - a - {bx}_{i} \vert$$

where yᵢ is the y coordinate for a plotted feature, xᵢ is the x coordinate for a plotted feature, b is the slope of the fitted line, and a is the y-intercept for the fitted line.

FIG. 26 illustrates the principle of the above-described minimization technique. FIG. 26 shows the same low-combined-intensity, central-trend features plotted with respect to the fitted line as in FIG. 25. FIG. 26 also shows the vertical distances, such as vertical distance 2602, of plotted features, such as plotted feature 2604, from the best-fit line 2606. The above-described technique minimizes the sum of these vertical distances. Another popular technique for line fitting is the least-squares technique. However, the least-squares technique is not the preferred method, as it is generally more sensitive to outlier data points than MAD-minimization techniques. Other alternatives include median-line fitting techniques. Note that alternative M quantities that can be minimized include the Euclidean distances of data points from the best-fit line, or the median Euclidean distance of data points from the best-fit line.

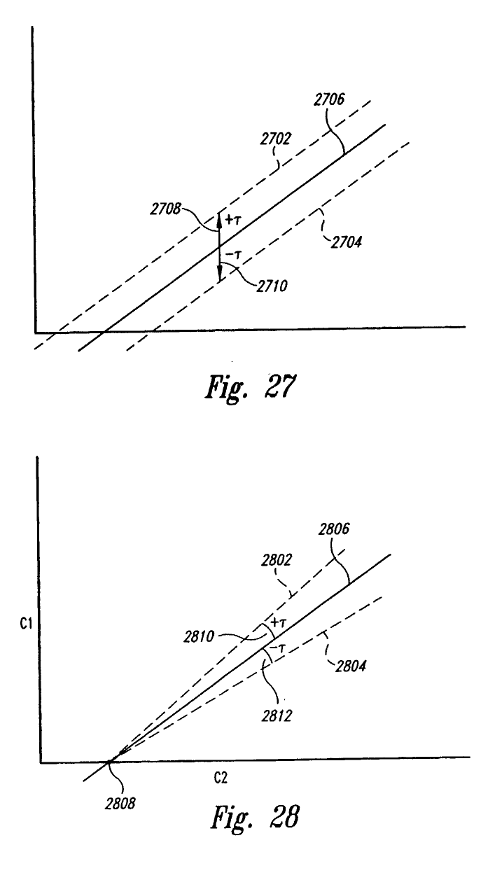

In step 1814, the line fit to the C1/C2 distribution for the low-combined-intensity, central-trend features is used to further filter this set of features based on their proximity, in the C1/C2 plot, to the best-fit line. FIGS. 27-28 illustrate two methods that can be employed to filter the low-combined-intensity, central-trend features with respect to the fitted line. In a first technique, illustrated in FIG. 27, two threshold lines 2702 and 2704 are constructed parallel with, and above and below, respectively, the fitted line 2706. The distance, in the vertical direction, between the fitted line 2706 and the threshold lines 2702 and 2704, τ, is related to the aggregate deviation of the plotted features in the C1/C2 plot from the best-fit line, or the value M minimized in the MAD technique described above. The threshold parameter τ may then be calculated by an expression such as:

$$τ = \sqrt{( ( \frac{1}{2} ) * {( kM )}^{2} )}$$

where k is a constant or a tunable parameter. Note that other estimators for the deviation of the plotted features from the fitted line can be substituted for M in the above equation. Note, as well, that other methods for determining the threshold parameter τ may be employed, and that τ itself may be a tunable parameter, rather than calculated from calculated deviations or error metrics for the plotted features. Once the two threshold lines 2702 and 2704 are constructed, it is straightforward to select those low-combined-intensity, central-trend features lying between the two threshold lines 2702 and 2704.

An alternative technique for filtering features based on proximity to the best-fit line is shown in FIG. 28. In this technique, rather than constructing parallel threshold lines, threshold lines with greater 2802 and lesser 2804 slopes than the best-fit line 2806 are constructed to fan out to either side of the best-fit line from the x-axis intercept 2808 or, in other cases, from the y-axis intercept, of the best-fit line. In this case, a threshold angle, τ, 2810 and 2812, can be computed based on a measure of the deviation of plotted features from the fitted line, or can be a tunable parameter. As in the previous threshold technique, those features lying between the threshold lines, or within a cone or hyper-cone, in higher dimensional cases, are chosen. Using either the filtering technique shown in FIG. 27 or that shown in FIG. 28, step 1814 produces a final set of filtered, non-control features.

The final set of filtered, non-control features is augmented, in step 1816, with non-saturated, uniform control features from the full data set, not initially selected in step 1804, that, when plotted in the C1/C2 plot, such as the C1/C2 plots shown in FIGS. 27 and 28, fall between the threshold lines and that also meet the x-percentile cutoff for features ranked with respect to combined intensities. In step 1818, the augmented, final filtered set produced in step 1816 is re-ranked with respect to combined feature intensities, as in step 1808, and a lowest percentile specified by the parameter y, similar to parameter x in step 1810, is selected as a characteristic set of background features. In an optional step 1820, this characteristic set can be further filtered using a box-plot filtering technique in which the highest quartile and the lowest quartile features with respect to combined intensities are removed from the characteristic background feature set. As a variation, box-plot analysis with fences is also possible. This essentially widens the scope of the box-plot by including features lying below and above a tunable parameter z times standard deviation of the distribution, in case of a uniform distribution, or z times the interquartile range, with respect to the 2^{nd} and 3^{rd} quartiles, respectively. Depending on stringency requirements, this increase in population of the features potentially enhances statistical accuracy.

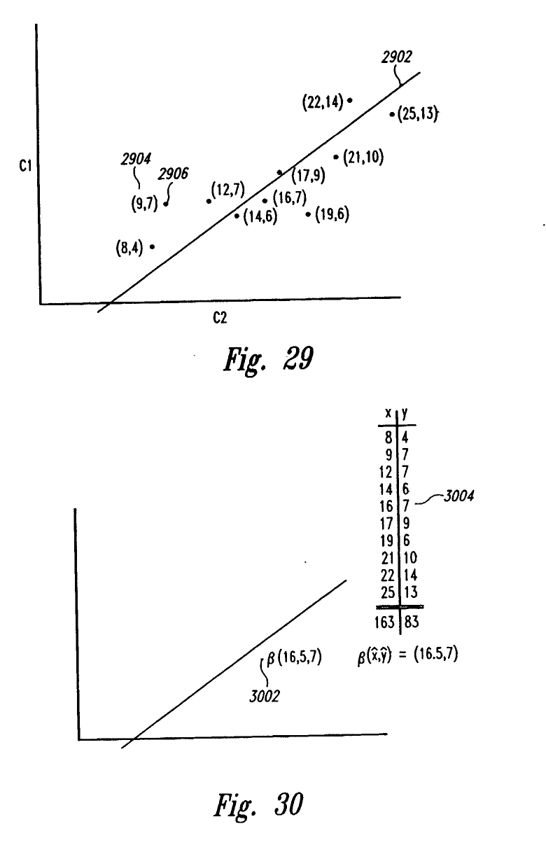

Next, in step 1822, an ideal background feature data point is constructed from the set of characteristic background features produced in step 1818 or 1820. An ideal background feature is a feature that accurately reflects the offsets in both channels that can be used to background correct a data set. FIGS. 29-30 illustrate the construction of an ideal background data point. In FIG. 29, the characteristic background features are plotted in a C1/C2 plot, distributed about the best-fit line 2902. In FIG. 29, the x,y coordinates for each feature are shown next to the plotted data point, such as the x,y coordinates (9,7) 2904 shown next to the plotted data point 2906. In FIG. 30, an ideal characteristic background feature β is constructed with x,y coordinates equal to the median x coordinate, calculated as the median of the x-coordinate values for the characteristic background features, and the median y coordinate, calculate as the median of the y-coordinate values of the characteristic background features. Thus, in FIG. 30, the ideal background features is plotted 3002 with x,y coordinates (16.5, 7) calculated from the x,y coordinates shown in the table 3004 in the upper right-hand comer of the figure. Note that the coordinates in table 3004 are sorted by x-coordinate value. Sorting by y-coordinate value would more directly reveal the median y-coordinate value 7. Alternatively, the ideal characteristic background feature β coordinates can be calculated as the average x-coordinate and y-coordinate values for the characteristic background features, or may be based on a percentile of characteristic-background-feature x-coordinate values and y-coordinate values or based on the xy-coordinates of a lowest characteristic background feature.

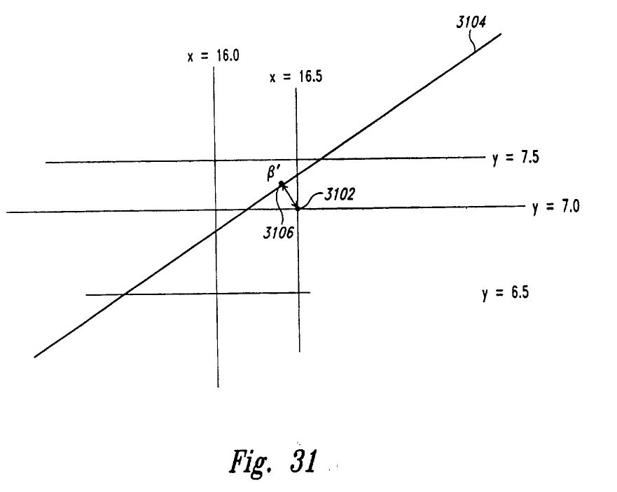

Next, in step 1824, the ideal characteristic background feature β is fuirther refined, or optimized, by projecting β back to the best fitted line. FIG. 31 illustrates a final refinement technique for calculating the ideal characteristic background feature. In FIG. 31, the data point plotted for the ideal characteristic background feature β 3102 is projected by translating a position of β in a direction perpendicular to the best-fit line 3104 to a position β′ 3106 coincident with the best-fit line 3104. The coordinates for the refined ideal characteristic background feature β′ can be calculated by solving simultaneous linear equations, one for the best-fit line with slope m, and one with slope −1/m that passes through the characteristic background feature β. Although step 1824 may be omitted in alternative embodiments, and the ideal characteristic background feature β used to compute the global, background-signal corrections, the refinement represented by step 1824 has proven to significantly increase the accuracy of the global, background-signal corrections. Finally, in step 1826, the x coordinate of the ideal characteristic background point is taken as the magnitude of the C2 background-signal correction, and the y coordinate of the ideal characteristic background point is taken as the magnitude of the C1 global background-signal correction, and both the C2 and C1 global background-signal corrections are applied to the entire data set. The net effect for the two-channel background correction is to move an ideal, lowest intensity-ratio data point (1606 in FIG. 16) to the origin of a C1/C2 distribution plot, rather than simply extending a best-fit curve to an x-axis or y-axis intercept, as discussed with reference to FIG. 13. It should be noted that the distance between the unprojected β and projected β′, as well as the difference between the mean and median values of β, may potentially serve as metrics of the quality of feature selection for the 2-channel background correction, and may serve as an error metric δ returned in step 1824.

### Matlab-Like Pseudocode Description of One Embodiment of the Present Invention

A Matlab-like pseudocode implementation of the above-described embodiment of the method of the present invention is provided below. This pseudocode implementation is provided for illustrative purposes, and is in no way intended to limit the scope of the current invention.

function [ UnProjected_Offset
Projected_Offset]=lowend3(lnumfeat,dcorrRG,dCutoff_pct_x,dMADMult,dCutoff_pct_y,

boxplotON);

if nargin < 1

lnumfeat = 3000; % number of features
dcorrRG = 0.05; %user defined correlation coefficient of feature in Red and green channels
dCutoff_pct_x = 0.2; % user settable parameter, with range between 0 to 1
dMADMult=0.73; % this parameter is user settable; Mult=0.73 is equivalent to 1.3SD
% This is a multiplier applied to the median/mean absolute deviation metric
% to define the region that is considered viable about the central tendency line
dCutoff_pct_y = 0.01; % user settable parameter, with range between 0 to 1
boxplotON = 0;

elseif nargin < 2

dcorrRG = 0.05;
dCutoff_pct_x = 0.2;
dMADMult=0.73;
dCutoff_pct_y = 0.01;
boxplotON = 0;

elseif nargin < 3

dCutoff_pct_x = 0.2;
dMADMult=0.73;
dCutoff_pct_y = 0.01;
boxplotON = 0;

elseif nargin < 4

dMADMult=0.73;
dCutoff_pct_y = 0.01;
boxplotON = 0;

elseif nargin < 5

dMADMult=0.73;
boxplotON = 0;

elseif nargin < 6

boxplotON = 0;

end
UnProjected_Offset = [0 0 0 0];
Projected_Offset = UnProjected_Offset;
% generate 2 channel data using a random number generator:
% and do an arbitrary scaling to 1000
dR=rand(lnumfeat,1)*1000;
dG=rand(lnumfeat,1)*1000;
% create feature array to denote initial candidate set
Feat_Array=[dR, dG];
siz=size(Feat_Array);
len_Feat_Array = siz(1);
dminIntensity =[0 0]; % initialize dminIntensity;
%this matrix will be used to check minimum intensity value in Rand G Space
%in case of Intensity <=0, the origin(0,0) for linear R and G intensity
%space will be transformed such that all intensity > 0
% Transpose Feat_Array from intensity Space to Rank Space
rankVect_R=ranktable(Feat_Array(:,1));
rankVect_G=ranktable(Feat_Array(:,2));
%[Feat_Array(:,1) rankVect_R Feat_Array(:,2) rankVect_G]
% Filter 1: Number of Correlated feats in Rank Space
INumCorrFeats = 0; % initialize number of correlated feats
factor=20;
dcorrRG
while INumCorrFeats<2

clear Corr_Feat; dminIntensity =[0 0]; INumCorrFeats = 0;
for feat_ctr=1:len_Feat_Array

dRho_R=find(rankVect_R==feat_ctr);
dRho_G=find(rankVect_G==feat_ctr);
RankCmp = abs(dRho_R-dRho_G)/len_Feat_Array;
if ( RankCmp <= dcorrRG)

INumCorrFeats=INumCorrFeats + 1;
Corr_Feat(INumCorrFeats)=feat_ctr;
dminIntensity =[(min(Feat_Array(feat_ctr,1), dminIntensity(1)))

(min(Feat_Array(feat_ctr,2), dminIntensity(2)))];

end

end
% relax the coupling criterion until at least 1 feature is selected
if (factor−5)>=1, dcorrRG=1/(factor−5); end

end
% Collapse from prior to new array based on Filter1
Feat_Array_1 =[Feat_Array(Corr_Feat(1:length(Corr_Feat)),1)

Feat_Array(Corr_Feat(1:length(Corr_Feat)),2)];

size(Feat_Array_1)
INumLinFitFeats=0;
if INumCorrFeats>1

INumCorrFeats
% Filter 2: 1st cutoff ceiling - this value can be user defined
% cutoff is performed on rankVect_RG
% Do ranking in RG Euclidean Distance space
% 1st generate Euclidean Distance Matrix; 2nd generate rank table
dGR_EucDist=gen_euclideanmatrix(Feat_Array_1,dminIntensity);
rankVect_RG=ranktable(dGR_EucDist), clear dGR_EucDist;
dCutoff_pctl_x = ceil(INumCorrFeats * dCutoff_pct_x)
INumCorrFeats_refined=0; % initialize number of refined correlated feats
for jj=1:INumCorrFeats

dRho_RG=find(rankVect_RG==jj);
if dRho_RG <= dOutoff_pctl_x

INumCorrFeats_refined=INumCorrFeats_refined + 1;
Corr_Feat_refined(INumCorrFeats_refined)=jj;

end

end
% Collapse from prior to new array based on Filter2
Feat_Array_2=[Feat_Array_1(Corr_Feat_refined,1) Feat_Array_1(Corr_Feat_refined,2)];

INumLinFitFeats = INumCorrFeats_refined % Number of feats used in the median fit
end
if INumLinFitFeats > 1

[dRGintercept,dRGslope,dLinFitMeanAbsDev]=Medfit(Feat_Array_2);
% Numerical Recipes Function, coded below
% dRGslope aught to give the correct R/G ratio, but need to correct for errors in
% noise-floor computation
% so expand envelope of acceptance by keeping slope=constant &
%varying intercept by LinFitMeanAbsDev
dTolerance = sqrt(0.5 *(dMADMult * dLinFitMeanAbsDev)\hat{ }2);
% defines the tolerance of the median envelope
dIntercept_envelope_lbound = (dTolerance * (dRGslope + 1)) + dRGintercept;
dIntercept_envelope_ubound = −(dTolerance * (dRGslope + 1)) + dRGintercept;
% Filter 3: All features that have a ratio that falls within the above defined
% intercept boundary
dminIntensity =[0 0]; % initialize dminIntensity
INumMedFeats = 0;
for jj=1:INumCorrFeats_refined

dRatioRG = Feat_Array_2(Corr_Feat_refined(jj),2)

/Feat_Array_2(Corr_Feat_refined(jj),1);

dInterceptRG = Feat_Array_2(Corr_Feat_refined(jj),2)-

(Feat_Array_2(Corr_Feat_refined(jj),1)* dRGslope);

%the following is based on a constant slope concept
if ((dRatioRG == dRGslope) | ((dInterceptRG <= dIntercept_envelope_lbound) &

(dInterceptRG >= dIntercept_envelope_ubound)))
INumMedFeats=INumMedFeats + 1;
Med_Feat(INumMedFeats)=jj;
% determine minimum intensity in each channel to ensure all feat intensity >0
dminIntensity =[(min(Feat_Array_2(jj,1),

dminIntensity(1))) (min(Feat_Array_2(jj,2), dminIntensity(2)))];

end

end
INumMedFeats
if length(Med_Feat) > 0

% Collapse from prior to new array based on Filter3
Feat_Array_3=[Feat_Array_2(Med_Feat,1) Feat_Array_2(Med_Feat,2)];
% Assuming 2 color data, make sure the origin(0,0) is such that all intensity > 0
dminIntensity=adjust_intensity_origin(dminIntensity);
% Filter 4: 2nd cutoff ceilling - this value can be user defined
% cutoff is performed on rankVect_RG2
dGR_EucDist=gen_euclideanmatrix(Feat_Array_3,dminIntensity);
rankVect_RG2=ranktable(dGR_EucDist); clear dGR_EucDist
dminIntensity =[0 0]; % initialize dminIntensity
dCutoff_pctl_y =ceil(INumMed Feats * dCutoff_pct_y)
INumMedFeats_refined=0;
for jj=1:INumMedFeats

dRho_RG=find(rankVect_RG2==jj);
if dRho_RG <= dCutoff_pctl_y

INumMedFeats_refined=INumMedFeats_refined + 1;
Med_Feat_refined(INumMedFeats_refined)=jj;
dminIntensity =[(min(Feat_Array_3(jj,1),

dminIntensity(1))) (min(Feat_Array_3(jj,2), dminIntensity(2)))];

end

end
INumMedFeats_refined
if length(Med_Feat_refined) > 0

Feat_Array_4=[Feat_Array_3(Med_Feat_refined,1)

Feat_Array_3(Med_Feat_refined,2)];

% Assuming 2 color data, make sure the origin(0,0) is such that all intensity > 0
dminIntensity=adjust_intensity_origin(dminIntensity);
% Filter 5: Box-plot analysis
if boxplotON % if box plot analysis is enabled

% First re-rank features in Euclidean Space
dGR_EucDist=gen_euclideanmatrix(Feat_Array_4,dminIntensity,

INumMedFeats_refined)

rankVect_RG3=ranktable(dGR_EucDist); clear dGR_EucDist
% detemine Inter Quartile Range
dRejectCutoff = [Round(INumMedFeats_refined * 0.25)

Round(INumMedFeats_refined * 0.75)];

for jj=1:INuMedFeats_refined

dRho_RG=find(rankVect_RG3==jj);
if (dRho_RG >= dLowRejectCutoff(1)) & (dRho_RG <= dLowRejectCutoff(2))

INumBoxPlotFeats=INumBoxPlotFeats + 1;
Boxplot_Feat(INumBoxPlotFeats)=jj;

end

end
if length(Boxplot_Feat)>0

Feat_Array_5=[Feat_Array_4(Boxplot_Feat(INumBoxPlotFeats,1))

Feat_Array_4(Boxplot_Feat(INumBoxPlotFeats,2))];

else

Feat_Array_5=Feat_Array_4;

end

else

Feat_Array_5=Feat_Array_4;

end
% Fill in stats for central tendency cluster
dMedian_R = median(Feat_Array_5(:,1));
dMedian_G = median(Feat_Array_5(:,2));
dMean_R = mean(Feat_Array_5(:,1));
dMean_G = mean(Feat_Array_5(:,2));
dSD_R = std(Feat_Array_5(:,1));
dSD_G = std(Feat_Array_5(:,2));
UnProjected_Offset = [ dMean_R dMean_G; dMedian_R dMedian_G];
% Determining the background offset - 2 ways
% project Median value of cluster back to Median Fit which is also the
%Central Tendency Line
dPerpendicularDist = abs((dRGslope * dMedian_R) − dMedian_G +

dRGintercept )/sqrt(dRGslope\hat{ }2 + 1);

dMedian_R_proj = dMedian_R − (dRGslope *

(dPerpendicularDist / (dRGslope\hat{ }2 + 1)));

dMedian_G_proj = dMedian_G + (1 * (dPerpendicularDist / (dRGslope\hat{ }2 + 1)));
% project Mean value of cluster back to Median Fit which is also the
% Central Tendency Line
dPerpendicularDist = abs((dRGslope * dMean_R) − dMean_G + dRGintercept)

/sqrt(dRGslope\hat{ }2 + 1);

dMean_R_proj = dMean_R − (dRGslope * (dPerpendicularDist / (dRGslope\hat{ }2 + 1)));
dMean_G_proj = dMean_G + (1 * (dPerpendicularDist / (dRGslope\hat{ }2 + 1)));
Projected_Offset = [ dMean_R_proj dMean_G_proj;

dMedian_R_proj dMedian_G_proj];

end

end

end
% FUNCTION:ranktable
function rankVect=ranktable(input)
% generate a rank table
[sorted_array, rankVect]=sortrows(input);
if length(rankVect)==1 & rankVect==1,

clear rankVect;
rankVect=(1:length(input)):

end
return;
% FUNCTION:gen_euclideanmatrix

function GR_EucDist=gen_euclideanmatrix(input,minIntensity)
% generate a Euclidean measure of features in R and G space
siz=size(input);
for ii = 1:siz(1)

GR_EucDist(ii)=sqrt((input(ii,1) + minIntensity(1))\hat{ }2 + (input(ii,2) + minIntensity(2))\hat{ }2);

end
return;

% FUNCTION:medFit
function [a,b,abdev]=Medfit(input)
% This function is from Numerical recipes
% Fits y=a+bx by the criterion of least absolute deviations
sr=0;
sg=0;
srg=0;
srr=0;
siz=size(input);
input_len=siz(1);
%finding the least squares fitting line
for m=1:input_len

temp_r(m)=input(m,1);
temp_g(m)=input(m,2);
sr=sr+temp_r(m);
sg=sg+temp_g(m);
srg=srg+(temp_r(m)*temp_g(m));
srr=srr+(temp_r(m)\hat{ }2);

end
% getting the least squares solutions
del=(input_len*srr)−(sr\hat{ }2);

| | |
|---|---|
| aa=((srr*sg)−(sr*srg))/del; | %least squares solutions |

bb=((input_len*srg)−(sr*sg))/del;
% the chi squares fit
chisq=0;
for n=1:input_len

chisq=chisq + ((input(n,2)) − (aa+(bb*input(n,1))))\hat{ }2;

end
% use standard deviation is an indicator of frquency/size of iteration steps
sigb=sqrt(chisq/del);
b1=bb;
[f1,abdev_tot]=rofunc(b1,input);
if f1>=0, b2=bb+(3.0*sigb);
else b2=bb+(−3.0*sigb);
end
[f2,abdev_tot]=rofunc(b2,input);
while (f1*f2 > 0.0)

bb=2.0*(b2−b1);
b1=b2;
f1=f2;
b2=bb;
[f2,abdev_tot]=rofunc(b2,input);
f1=f1
f2=f2

end
sigb=0.01*sigb;
while (abs(b2−b1) > sigb)

bb=0.5*(b1+b2);

if (bb == b1 | bb == b2) break; end
[f,abdev_tot]=rofunc(bb,input);
if (f*f1 >= 0.0)

f1=f;

b1=bb;

else

f2=f;
b2=bb;

end

end
a=aa;
b=bb;
abdev=abdev_tot/input_len;
return;
% FUNCTION:rofunc
function [out_sum,abdev_tot]=rofunc(inval, input)
warning off;
siz=size(input);
n1=siz(1)+1;
nml=n1/2;
nmh=n1−nml;
siz=size(input);
input_len=siz(1);
for kk=1:input_len

arr(kk)=input(kk,2)−inval*input(kk,1);

end
sorted_arr=sort(arr);
aa=0.5*(arr(nml)+arr(nmh));
sum_val=0;
abdev_tot=0;
for ll=1:input_len

d=input(ll,2)−(inval*input(ll,1)+aa);
abdev_tot = abdev_tot +abs(d);
if d>=0, sum_val=sum_val+input(ll,1)*1.0;
else sum_val=sum_val+input(ll,1)*(−1.0);
end

end
out_sum=sum_val;
return;
%FUNCTION: Adjust_IntensityOrigin
function adj_minIntensity=adjust_intensity_origin(minIntensity)
% Assuming 2 color data, make sure the origin(0,0) is such that all intensity > 0
if (minIntensity(1) <= 0.0)

adj_minIntensity(1) = 1.0 − minIntensity(1);

else

adj_minIntensity(1) = 0.0;

end
if (minIntensity(2)<= 0.0)

adj_minIntensity(2) = 1.0 − minIntensity(2);

else

adj_minIntensity(2)= 0.0;

end
return;

Although the present invention has been described in terms of a particular embodiment, it is not intended that the invention be limited to this embodiment. Modifications within the spirit of the invention will be apparent to those skilled in the art. For example, the method and system can be straightforwardly extended to determine global background-signal corrections for multi-channel data sets including signals for more than two channels. In one approach, a hyper-dimensional signal-intensity space is considered, each dimension corresponding to a particular channel, and a best-fit hyper-volume calculated for the distribution of feature intensities in the hyper-dimensional signal-intensity space. The lowest-combined-intensity features can then be selected to compute the hyper-dimensional coordinates of an ideal characteristic background data point, from which global background-signal corrections for each channel can be obtained. Alternatively, global background-signal corrections can be calculated iteratively by repeatedly calculating, in a pair-wise fashion, relative global background-signal corrections for pairs of channels. The global background-signal correction method can be implemented in an almost limitless number of ways, in many different computer languages for execution in many different types of computers, using an almost limitless number of different modular organizations, control structures, data structures, and variables. Different sub-methods can be employed. For example, rather than using the rank-ordering method for discovering central-trend features, a geometric or statistical method may be employed. As discussed above, different methods for fitting a line or curve to data-point distributions can be employed. Although each step shown in FIG. 18 is carried out in a preferred embodiment of the present invention, alternative embodiments may omit one or more individual steps shown in FIG. 18, and a workable, background-signal correction method can still obtained, due to the robust nature of the method described in FIG. 18. For example, either or both of steps 1808 and 1824 may be omitted, and the resulting alternative embodiments still produce useful background-signal corrections. Global, background-signal corrections calculated by an embodiment of the method of the present invention can be used to evaluate operation of a molecular array scanner, calibrate a molecular array scanner, evaluate the quality of a molecular array, evaluate the correctness of background subtraction methods and other data processing methods, and evaluate of the reproducibility of a molecular-array-based experiment. For example, background-signal corrections calculated by an embodiment of the method of the present invention can be compared against standard or expected background-signal corrections, and, if the observed background-signal corrections fall outside the standard or expected ranges, molecular arrays exceeding or failing to meet standards or expectations may be rejected or remanufactured, scanners producing data with observed background-signal corrections falling outside the standard or expected range may be recalibrated, adjusted, or partially remanufactured, and molecular array experiments producing data with observed background-signal corrections falling outside the standard or expected range may be redesigned or repeated. The present invention further provides a computer program product that may execute a method of the present invention. The program product includes a computer readable storage medium having a computer program stored thereon and which, when loaded into a programmable processor, provides instructions to the processor of that apparatus such that it will execute the procedures required of it to perform a method of the present invention. The storage medium can, for example, be a portable or fixed computer readable storage medium, whether magnetic, optical or solid state device based.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for calculating global, background-signal corrections for a multi-channel, molecular-array data set, the method comprising:
selecting a set of low-combined-intensity features from the data set;
determining a position of a characteristic background data point based on the selected, low-combined-intensity features in a signal-intensity space with dimensions corresponding to the channels; and
determining global, background-signal corrections for each channel from the position of the characteristic background data point within the signal-intensity space.

2. The method of claim 1 wherein selecting a set of low-combined-intensity features from the data set further includes:
selecting non-control features from the data set;
filtering the selected non-control features to remove non-uniform features and signal-saturated features;
selecting central-trend features from the filtered, selected non-control features; and
selecting a lowest-intensity percentile subset of the selected central-trend features.

3. The method of claim 2 wherein selecting central-trend features from the filtered, selected non-control features further includes;
ordering each channel-specific data subset within the data set by feature intensity; and
selecting as central-trend features those features with identical or similar ranks in all channels.

4. The method of claim 2 wherein selecting a set of low-combined-intensity, central-trend features from the data set further includes:
determining a best-fit representation to describe the central-trend of features distributed within the signal-intensity space; and
selecting features proximal to the best-fit representation in signal-intensity space.

5. The method of claim 4 wherein determining a best-fit representation to describe the central trend of features further includes:
constructing a curve, volume, or hyper-volume for two-channel, three-channel, and more-than-three-channel data sets, respectively, that represents the central trend of features distributed within the signal-intensity space.

6. The method of claim 4 wherein selecting a set of low-combined-intensity, central-trend features from the data set further includes:
augmenting the set of features proximal to the best-fit representation in signal-intensity space with control features of low intensity proximal to the best-fit representation in signal-intensity space.

7. The method of claim 1 wherein calculating global, background-signal corrections for each channel from the position of the characteristic background data point within the signal-intensity space further includes:
for each channel, selecting the magnitude of the coordinate of the characteristic background data point with respect to the channel in the signal-intensity space as the global, background-signal correction for the channel.

8. The method of claim 1 further including:
applying the global, background-signal correction for each channel to the data set by adding the global, background-signal correction to the feature intensities within the data subset corresponding to the channel.

9. A representation of a background-corrected data set, produced using the method of claim 8, that is maintained for subsequent analysis by one of:
storing the representation of the background-corrected data set in a computer-readable medium; and
transferring the representation of the background-corrected data set to an intercommunicating entity via electronic signals.

10. Results produced by a molecular-array data processing program employing the method of claim 8 stored in a computer-readable medium.

11. Results produced by a molecular-array data processing program employing the method of claim 8 printed in a human-readable format.

12. Results produced by a molecular-array data processing program employing the method of claim 8 transferred to an intercommunicating entity via electronic signals.

13. A method according to claim 8 wherein the background signal intensity is communicated to a remote location.

14. A method comprising receiving data produced by using the method of claim 8.

15. The method of claim 1 wherein a multi-channel, molecular-array data set includes:
a data set containing data subsets corresponding to feature signals obtained from scanning a single molecular array in two or more different channels;
a data set containing data subsets corresponding to feature signals obtained from scanning two or more different arrays in a single channel; and
a data set containing data subsets corresponding to feature signals obtained from scanning two or more different arrays in two or more different channels.

16. Using one or more global background-signal corrections calculated by the method of claim 1 to carry out one of:
evaluation operation of a molecular array scanner;
evaluation of the quality of background correction;
evaluation of the quality of data corrections other than background corrections;
calibration a molecular array scanner;
evaluation the quality of a molecular array; and
evaluation of the reproducibility of a molecular-array-based experiment.

17. A computer program including an implementation of the method of claim 1 stored in a computer readable medium.

18. A method comprising forwarding data produced by using the method of claim 1.

19. A multi-channel, molecular-array data-set processing system comprising:
a computer processor;
a communications medium by which molecular-array data points are received by the molecular-array-data processing system;
one or more memory components that store molecular-array data points; and
a program, stored in the one or more memory components and executed by the computer processor, that:
selecting a set of low-combined-intensity features from the data set;
determining a position of a characteristic background data point based on the selected, low-combined-intensity features in a signal-intensity space with dimensions corresponding to the channels; and
determining global, background-signal corrections for each channel from the position of the characteristic background data point within the signal-intensity space.
